(19) United States
(12) Patent Application Publication
(10) Pub. No.: US 2010/0221768 A1
(43) Pub. Date: Sep. 2, 2010

(54) CELL CULTURE DISH

(75) Inventors: Tomonori Akai, Tokyo (JP); Yasuhito Onishi, Tokyo (JP); Kei Imai, Fukushima (JP); Yoshio Aikawa, Fukushima (JP); Masaki Ohtake, Fukushima (JP); Tamas Somfai, Ibaraki (JP)

Correspondence Address: SUGHRUE MION, PLLC, 2100 PENNSYLVANIA AVENUE, N.W., SUITE 800, WASHINGTON, DC, 20037, US

(73) Assignees: Dai Nippon Printing Co., Ltd., Tokyo (JP); National Livestock Breeding Center, Nishishirakawa-gun (JP)

(21) Appl. No.: 12/702,526

(22) Filed: Feb. 9, 2010

(30) Foreign Application Priority Data
Feb. 9, 2009 (JP) 2009-027845
Feb. 3, 2010 (JP) 2010-022463

(51) Int. Cl. C12Q 1/02; C12M 1/22

(52) U.S. Cl. 435/29; 435/305.2

(57) ABSTRACT

An object of the present invention is to provide a culture dish appropriate for automatically identifying cultured cells. The present invention relates to a culture dish for culturing cells that require separate control, which has a bottom wall and a side wall, wherein cell-holding parts having wells are arranged on the bottom wall, 4 or more wells are adjacent to each other, the wall surface of each well has a concave surface that slopes upward from the lowest position to the outer edge of the well, and the pitch between adjacent wells is 1 mm or less.

### FIELD OF THE INVENTION

The present invention relates to a culture dish for culturing cells that require separate control, such as embryos, and a method for identifying cultured cells using the culture dish.

### BACKGROUND OF THE INVENTION

Embryos (zygotes) are produced by in vitro fertilization of sperm and oocytes in a culture system. The embryos are then cultured to a stage of hatched blastocysts having hatched from the zona pellucida via the stages of cleavage, morula, and then blastocyst. As a result of realization of these techniques, assisted reproductive technology (ART) has been established not only in the field of domestic animals, but also in the field of medical care for human infertility, which involves implantation of the embryos at a stage between cleavage and blastocyst into the uterus, so as to obtain infants.

However, in vitro fertilization does not always result in a high pregnancy success rate. For example, the pregnancy success rate of human in vitro fertilization still remains at a level between about 25% and 35%. A reason for this is the low possibility of obtaining high-quality embryos suitable for implantation into the uterus by culture. Whether or not cultured embryos are high-quality embryos suitable for implantation into the uterus is identified by specialists who observe each embryo via a microscope. Therefore, distinguishing high-quality embryos from other embryos is problematic in that it requires much cost and time. Hence, technology has been required that allows the distinguishing of high-quality embryos suitable for implantation into the uterus from other embryos at low cost within a short time. There is also a problem such that identification of high-quality embryos differs among different individuals. Criteria for identifying high-quality embryos and technology for selecting such high-quality embryos have been required.

Conventionally, embryos have been cultured by a method that involves adding approximately 500 μL of a culture solution into wells on a culture dish and then culturing embryos in the culture solution, placing approximately 20 μL of microdroplets within a well on the culture dish, coating the surfaces of the microdroplets with mineral oil, and then placing embryos thereinto (Sugawara, Ogawa (Ed.), “Method for Culturing Cells with Reproductive Functions,” Japan, Japan Scientific Societies Press, Published June, 1993, pp. 25-153), for example. Also, M. Taka, et al. Journal of Reproduction and Development, 51, 533-537 (2005); and Vajta G., et al. Molecular Reproduction and Development, 55, 256-264 (2000) disclose a method for separate control of embryos that involves providing a plurality of wells on a culture dish, introducing an embryo into each well, and then culturing the embryos. P. J. Booth, et al. Biology of Reproduction, 77, 765-779 (2007) discloses a method for separate control of embryos that involves arranging a mesh network on the culture dish, introducing an embryo into each separate portion of the mesh, and then culturing the embryos.

In recent years, research and development regarding cell therapy and/or regenerative medicine have advanced, so that needs are increasing concerning maintenance of a sterile environment during cell culture, cell quality control, or rapid and mass culture of cells and determining differences among the same. However, conventionally, the state of cultured cells has been observed manually using culture dishes as described in the above documents. Such conventional techniques pose problems in that: continuous observation of the state of cells is difficult; there is concern that cells may be affected by changes in culture environment and contamination risk due to saprophytes, for example; and identification of cultured cells involves high costs and much time, resulting in poor efficiency, for example. Therefore, development of technology for automatically identifying cultured cells has been desired. Also, for example, in the field of animal industry, the government has set up “A Goal for Improvement and Growth of Domestic Animals” (“Kachiku Kairyo Zoshoku Mokuhyou”). To achieve this goal, an increase in the number of domestic animals of special species used for meat has been promoted utilizing implantation of embryos. Under such circumstances, automatic identification technology for cells is very useful.

### SUMMARY OF THE INVENTION

In view of the above conventional circumstances, the present inventors have examined the possibility of automatic identification of embryos in order to identify high-quality embryos suitable for implantation into the uterus at a low cost within a short time. As a result of examination of such automation using the culture dish described in Sugawara, Ogawa (Ed.), “Method for Culturing Cells with Reproductive Functions,” Japan, Japan Scientific Societies Press, published June 1993, pp. 25-153, a plurality of embryos moved within plates or microdroplets during culture, following which embryos having different qualities could not be individually controlled, revealing that such technique is unsuitable for automatic identification of embryos. Also, embryos may be automatically identified by taking photographic images of embryos using a CCD camera or the like. However, it has also been revealed that taking photographic images of individual embryos is difficult with the use of a conventional culture dish.

Accordingly, the present inventors have examined automation using culture dishes provided with a plurality of depressions (wells) as described in M. Taka, et al. Journal of Reproduction and Development, 51, 533-537 (2005); Vajta G., et al. Molecular Reproduction and Development, 55, 256-264 (2000); and P. J. Booth, et al. Biology of Reproduction, 77, 765-779 (2007) or culture dishes with mesh networks arranged thereon. Thus, the present inventors have discovered that separate control of embryos is possible because of the high density of embryos. They have also discovered that extraction of embryo images by outline extraction for images taken is difficult when the embryos move within each well or each mesh portion of the mesh network to come into contact with a side wall of the relevant well or the mesh portion.

Therefore, an object of the present invention is to provide a culture dish appropriate for automatically identifying cultured cells.

As a result of examination to address the above problems, the present inventors have discovered that cell outline extraction can be efficiently carried out for photographic images of cultured cells by culturing cells using a culture dish that has a cell-holding part(s). In the cell-holding part(s), wells are densely arranged and each well has a concave surface that slopes upward from the lowest position to the outer edge of the well. Thus, the present inventors have discovered that such culture dish is appropriate for automatic identification of cultured cells and have completed the present invention.

The present invention encompasses the following (1) to (18).

(1) A culture dish for culturing cells that require separate control, having a bottom wall and a side wall, wherein:
a cell-holding part having wells is arranged on the bottom wall;
4 or more wells are adjacent to each other;
the wall surface of each well has a concave surface that slopes upward from the lowest position to the outer edge of the well; and
the pitch between wells adjacent to each other is 1 mm or less.
(2) The culture dish according to (1), wherein the wall surface of each well has a concave surface having a straight section.
(3) The culture dish according to (2), wherein the surface roughness of the concave surface is characterized in that the maximum height Ry is less than 1.0 μm.
(4) The culture dish according to any one of (1) to (3), wherein the width of the opening of each well ranges from 100 μm to 300 μm.
(5) The culture dish according to any one of (1) to (4), wherein the depth of each well ranges from 50 μm to 200 μM.
(6) The culture dish according to any one of (1) to (5), wherein the opening of each well is circular.
(7) The culture dish according to (6), wherein the wall surface of each well has a conical or circular cone-shaped part.
(8) The culture dish according to (7), wherein an angle formed by the center line and the bus line of the conical or circular cone-shaped part ranges from 89° to 45°.
(9) The culture dish according to any one of (1) to (8), wherein the wells adjacent to each other are arranged at a density of at least one well per 1 mm².
(10) The culture dish according to any one of (1) to (9), wherein 4 or more wells adjacent to each other are arranged in the form of a square grid or close packing.
(11) The culture dish according to any one of (1) to (10), wherein 24 or more wells are arranged.
(12) The culture dish according to any one of (1) to (11), wherein the cell is selected from the group consisting of an embryo, an ovum, an ES cell, and an iPS cell.
(13) The culture dish according to (12), wherein the cell is a bovine embryo.
(14) The culture dish according to any one of (1) to (13), wherein 4 or more wells adjacent to each other are separated by an inner wall surrounding the wells from the other part within the culture dish.
(15) The culture dish according to (14), having a liquid-holding part that is a peripheral part of the culture dish having no cell-holding part.
(16) The culture dish according to any one of (1) to (15), which is used for automatically identifying cultured cells.
(17) The culture dish according to (16), regarding which a visual field for observation contains 4 or more wells when a cell-holding part is observed via a microscope using a 4× objective lens.
(18) A method for identifying cultured cells, comprising

introducing and culturing cells selected from the group consisting of embryos, ova, ES cells, and iPS cells in wells of a cell-holding part of the culture dish according to any one of (1) to (17),

taking photographic images of cultured cells obtained via a microscope using a detector, and

subjecting the thus obtained images to outline extraction.

### EFFECT OF THE INVENTION

According to the present invention, a culture dish appropriate for automatic identification of cultured cells is provided.

### BRIEF DESCRIPTION OF THE DRAWINGS

FIG. 1a schematically shows an embodiment of the present invention.

FIG. 1b schematically shows an embodiment of the present invention.

FIG. 2a schematically shows a culture dish A that is an embodiment of the present invention.

FIG. 2b schematically shows an embodiment of the present invention.

FIG. 3 is a plain view where wells adjacent to each other are arranged in the form of close packing.

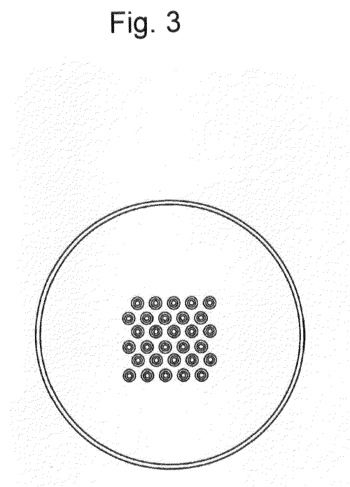

FIG. 4 shows a culture dish B used in Comparative example.

FIG. 5 shows a microscopic photograph showing an embryo cultured in the culture dish A of the present invention.

FIG. 6 shows the result of threshold-processing for the image of FIG. 5.

FIG. 7 shows a microscopic photograph showing an embryo in the culture dish B used in Comparative example.

FIG. 8 shows the result of threshold-processing for the image of FIG. 7.

FIG. 9 shows a microscopic photograph showing an embryo cultured in the culture dish B used in Comparative example.

FIG. 10 shows the result of threshold-processing for the image of FIG. 9.

FIG. 11 is a plain view showing a culture dish C that is an embodiment of the present invention.

FIG. 12 is a IIIa-IIIa (FIG. 11) cross-sectional view.

FIG. 13 is a partially enlarged view of the cell-holding part in FIG. 11.

FIG. 14 is a IIIb-IIIb (FIG. 13) cross-sectional view.

FIG. 15 is a partially enlarged view of a well in FIG. 13.

FIG. 16 is a IIIc-IIIc (FIG. 15) cross-sectional view.

FIG. 17 shows a photographic image showing the appearance of the culture dish C.

FIG. 18 shows a graph showing the results of measuring the cross-sectional shape of the cell-holding part of the culture dish C.

FIG. 19 shows a graph showing the results of measuring cross-sectional shapes of the wells of the culture dish C.

FIG. 20 shows the results of correcting the slope through linear fitting for the concave surface on the right (FIG. 19).

FIG. 21 shows a graph showing the results of measuring cross-sectional shapes of the wells of the culture dish D.

FIG. 22 shows the results of correcting the slope through linear fitting for the concave surface on the right (FIG. 21).

FIG. 23 is a plain view showing a culture dish E that is an embodiment of the present invention.

FIG. 24 is a IVa-IVa (FIG. 23) cross-sectional view.

FIG. 25 is a partially enlarged view showing the cell-holding part in FIG. 23.

FIG. 26 is an IVb-IVb (FIG. 25) cross-sectional view.

FIG. 27 is a partially enlarged view showing the wells in FIG. 25.

FIG. 28 is an IVc-IVc (FIG. 27) cross-sectional view.

FIG. 29 is a graph showing the results of measuring cross sectional shapes of the wells of the culture dish E.

FIG. 30 shows the results of correcting the slope through linear fitting for the concave surface on the right (FIG. 29).

FIG. 31 is a microscopic photograph showing a mouse embryo cultured in the culture dish C of the present invention.

FIG. 32 shows the result of threshold-processing for the image of FIG. 31.

FIG. 33 is a microscopic photograph showing a bovine embryo cultured in the culture dish C of the present invention.

FIG. 34 shows the result of threshold-processing for the image of FIG. 33.

### DETAILED DESCRIPTION OF THE PREFERRED EMBODIMENTS

The present invention relates to a culture dish having a bottom wall and a side wall for culturing cells that require separate control, wherein:

a cell-holding part having wells is arranged on the bottom wall;

4 or more wells are adjacent to each other;

the wall surface of each well has a concave surface that slopes upward from the lowest position to the outer edge of the well; and

the pitch between wells adjacent to each other is 1 mm or less.

The term “cells that require separate control” refers to cells that should be individually specified during and after culture, since confusion thereof with each other in a culture dish where a plurality of cells are cultured is not desired. Examples of such cells that require separate control include embryos, ova, ES cells (embryonic stem cells), and iPS cells (induced pluripotent stem cells). The term “ova” refers to unfertilized ova and examples thereof include immature oocytes and mature oocytes. After fertilization, an embryo increases in the number of cells by cleavage from 2-cell to 4-cell and then to 8-cell stages and is then developed into a morula and then into a blastocyst. Examples of embryos include early embryos such as a 2-cell embryo, a 4-cell embryo, and a 8-cell embryo, morulae, and blastocysts (including an early blastocyst, an expanded blastocyst, and hatched blastocyst). The term “blastocyst” refers to an embryo comprising trophoblastic cells having potential ability of forming a placenta and an inner cell mass having potential ability of forming a fetus. The term “ES cells” refers to undifferentiated multipotent or totipotent cells obtained from inner cell masses of blastocysts. The term “iPS cells” refers to cells to which ES-cell-like versatility for differentiation has been conferred via introduction of several types of gene (transcriptional factors) into somatic cells (mainly fibroblasts). Specifically, examples of cells to be cultured in the present invention also include aggregates of a plurality of cells, such as embryos or blastocysts. The culture dish of the present invention is suitable for culturing preferably mammalian cells and avian cells and particularly suitable for culturing mammalian cells. The term “mammals” refers to warm blooded vertebrates and examples thereof include primates such as humans and monkeys, rodents such as mice, rats, and rabbits, pet animals such as dogs and cats, and domestic animals such as cattle, horses, and pigs. The culture dish of the present invention is particularly suitable for culturing bovine embryos.

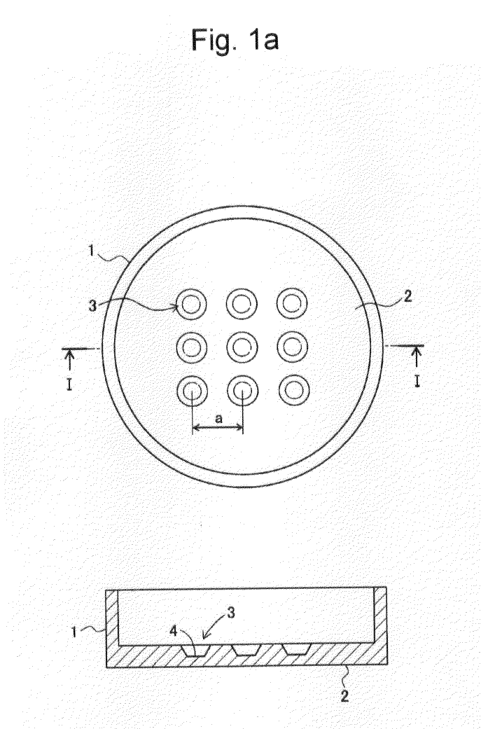

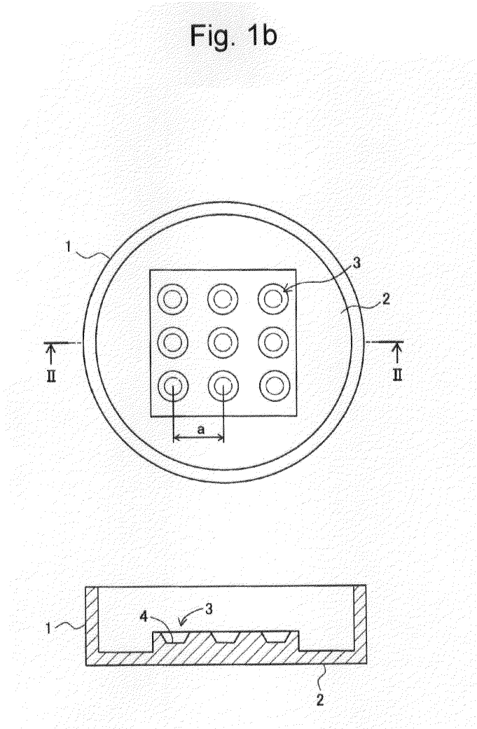

The culture dish of the present invention has a bottom wall and a side wall and can hold liquid within a space formed by the bottom wall and the side wall. The shape of such bottom wall is not particularly limited and may be polygonal such as triangle and quadrangle or circular (including circular and elliptic shapes). The side wall is formed surrounding the outer edge of the bottom wall. In the culture dish of the present invention, an opening is generally provided opposite to the bottom wall. In addition, the culture dish of the present invention may have a lid similar to that of a general petri dish. The bottom wall and the side wall of the culture dish of the present invention preferably form a shape similar to a conventionally used petri dish. Furthermore, cell-holding parts having wells are formed on the bottom wall of the culture dish. Wells of the cell-holding parts may be directly provided as wells on the bottom wall of the culture dish (FIG. 1a) or formed with members protruding from the bottom wall (FIG. 1b).

Four (4) or more, preferably 6 or more, and more preferably 8 or more wells are formed adjacent to each other. At least 4 wells are formed adjacent to each other and other wells may be separately formed not adjacent to each other. Also, a plurality of groups each consisting 4 or more wells formed adjacent to each other may be arranged and the groups may be not adjacent to each other.

Each well has a wall surface and an opening. The shape of the outer edge of the opening is not particularly limited and is preferably circular (including circular and elliptic shapes).

The pitch between wells adjacent to each other is 1 mm or less. However, the pitch differs depending on the types of cells to be held.

For example, when wells hold bovine embryos, the pitch between wells adjacent each other is generally 1 mm or less, preferably 0.7 mm or less, and further preferably 0.45 mm or less. An observation apparatus that is often used is provided with a ½-inch CCD element, a 4× objective lens, a 10× objective lens, and a 20× objective lens. An observable visual field is approximately 1.6 mm×1.2 mm when a 4× objective lens is selected upon the use of such observation apparatus. It is preferably designed so that 4 or more wells are contained within the visual field for observation. Suppose the diameter of a well is 0.3 mm, which is greater than the diameter (0.25 mm) of a bovine embryo. When the pitch is 0.9 mm or less, 4 (=2×2) embryos can be observed within a microscopic field of 1.6 mm×1.2 mm. When the pitch is 0.65 mm or less, 6 (=3×2) embryos can be observed within the microscopic field of 1.6 mm×1.2 mm. When the pitch is 0.43 mm or less, 12 (=4×3) embryos can be observed within the microscopic field of 1.6 mm×1.2 mm. Four or more wells are designed to be contained within the visual field for observation, so that multiple cells can be treated at once when cultured cells are automatically identified, for example. Also, comparison and the distinguishing of one embryo from the other embryos within the visual field are facilitated. Hence, identification can be carried out more efficiently.

The pitch between wells is a distance between the center of a well and the center of a well adjacent thereto (FIG. 1a). The term “the center of a well” refers to the position of the center of gravity of a figure formed by the outer edge of the opening of the well or the center of a circle when the outer edge is circular. The term “pitch between wells” generally refers to a mean pitch. Such mean pitch is calculated from pitches between a well and all wells adjacent to the well. The pitch between wells is greater than the outer edge size of the opening of a well. The term “outer edge size of the opening of a well” refers to a diameter of the outer edge of the opening when the outer edge is circular or otherwise refers to a mean value (calculated from the greatest and the shortest diameters) of a figure formed by the outer edge of the opening. That is, wells adjacent to each other are arranged at a density of at least 1 well and preferably at least 4 wells per mm². Four (4) or more wells adjacent to each other are preferably arranged in the form of a square grid or close packing. An example is a case in which 25 wells are arranged in the form of a 5×5 square grid. Arrangement in the form of a square grid or close packing facilitates specification of the position of each well on the bottom wall of the culture dish and its application to automation. Also, preferably 24 or more wells in total and more preferably 50 or more wells in total are arranged per culture dish. Provision of many wells per culture dish can decrease the number of culture dishes required for culturing a certain number of cells, which is economically advantageous.

When the outer edge of the opening of each well is circular, the pitch between wells adjacent to each other can also be specified by X+m+n (where, X denotes the greatest diameter of cells, m denotes a length found by subtracting the greatest diameter of cells from the diameter of a circle formed by the outer edge of the opening of a well, and n denotes the length of a partition between wells). The term “partition between wells” refers to the shortest distance between the outer edges of wells adjacent to each other. Here, m is generally 0.1 mm or less, preferably 0.07 mm or less, and further preferably 0.05 mm or less and n is generally 0.6 mm or less, preferably 0.35 mm or less, and further preferably 0.15 mm or less.

Wells are densely arranged at the above pitch, so that many cells can be cultured simultaneously while controlling cells individually. Furthermore, one microscopic field can contain many cells, so that an image of many cells can be obtained at once.

An opening of a well of a cell-holding part has the width sufficient for holding cells. Here, the term “the width of the opening of a well” refers to the length of the shortest diameter of a figure formed by the outer edge of the opening of a well. Therefore, when the outer edge of the opening of a well is circular, the width of the opening equals the diameter of the circle and the diameter is greater than the maximum size of cells to be cultured. When an embryo is cultured using the culture dish of the present invention, it is desirably cultured to the stage of blastocyst. Hence, the diameter of a circular opening is desirably greater than the maximum size of cells at the stage of blastocyst. The maximum size of cells at the stage of blastocyst generally ranges from 100 μm to 280 μm, so that the diameter of a circular opening is generally 100 μm or more. Also, as described above, the size of the outer edge of the opening of a well (mean value of the greatest and the shortest diameters of a figure formed by the outer edge of the opening) is lower than the pitch between wells. When the outer edge of an opening is circular, the diameter of the circle is shorter than the pitch between wells; that is, shorter than the distance between the centers of circles, which is generally less than 1 mm. The above width and size of an opening differ depending on the types of cells to be held. In general, the width of the opening of a well preferably ranges from 100 μm to 300 μm, for example.

For example, in the case of a bovine embryo, the width of the opening of a well (diameter when the well is circular) is generally 0.25 mm or more, preferably 0.26 mm or more, further preferably 0.27 mm or more, generally less than 1 mm, preferably less than 0.7 mm, and further preferably less than 0.45 mm or less. Also, the width of the opening of the above well can also be specified by X+m (where X denotes the greatest diameter of cells). Here, m is preferably 0.01 mm or more, and further preferably 0.02 mm or more.

The wall surface of each well of a cell-holding part has a concave surface that slopes upward from the lowest position to the outer edge of the well. Wells of a cell-holding part are each generally formed so that the bottom is placed on the bottom wall side of the culture dish. Regarding the shape (profile) of the concave surface of each well, the following cases can be adequately employed, such as a case in which the concave surface slopes or curved upward from the lowest position to the outer edge of the well, a case in which the concave surface slopes upward in a staircase pattern. In particular, the concave surface preferably contains a straight section; that is, the path entirely or partially slopes upward linearly from the lowest position to the outer edge of the well. Containment of such straight section suppresses the movement of cells arranged within each well and facilitates fixation of the cells at the deepest position within the well. Therefore, clear images can be obtained when cells are observed via a microscope.

With high surface roughness of the concave surface, a clear outline may not be able to obtain due to concavity and convexity on the concave surface when an image (that had been subjected to transmission observation via a microscope) is subjected to outline extraction. Surface roughness is preferably as low as possible. Specifically, the maximum height Ry (refers to a distance between the mountaintop line and the valley floor of a portion with only a reference length extracted from a roughness curve into the direction of the average line) is preferably less than 1.0 μm and is particularly preferably less than 0.5 μm. In addition, the surface roughness of the concave surface can be lowered by increasing the processing accuracy of a template through polishing or the like upon production of a template for the culture dish, for example.

The wall surface of a well of a cell-holding part preferably contains a conical or circular cone-shaped portion. Such conical or circular cone-shaped portion is formed, so that the top of the cone, or the upper or the lower surface (of the circular cone) having a smaller area than the other is placed on the bottom wall side of the culture dish. Examples of such cone include a cone, an elliptic cone, and shapes analogous thereto, such as a cone or an elliptic cone with a rounded top, a shape with a conical surface laterally swollen outward, and a shape with a conical surface dented inward. Examples of such circular cone include a circular cone, an elliptic cone, and shapes analogous thereto. Specific examples thereof include a shape in which a junction between the upper or lower surface of a circular cone or an elliptic cone and the conical surface is rounded, a shape in which the conical surface is swollen outward, and a shape in which the conical surface is dented inward. When the wall surface of a well of a cell-holding part contains a circular cone-shaped portion, the diameter of the upper or lower surface (of the circular cone) having a smaller area than the other, which is placed on the bottom wall side of the culture dish is preferably lower than the greatest diameter of cells. Specifically, the diameter of such surface having a smaller area than the other is preferably a half of or less than the greatest diameter of cells. The size that is a half size or less than the greatest diameter of cells facilitates arrangement of cells at the center of a well, suppresses movement, and facilitates taking photographic images of cells. Also, particularly the diameter of such surface having a smaller area than the other is preferably 10 μm or less. The diameter of 10 μm or less is effective since it narrows the central region that is observed with difficulty when affected by refraction of light, scattering and the like of light upon taking photographic images, so that clear images can be obtained. In addition, the conical surface of the conical or circular cone-shaped portion of a well is a curved surface and preferably contains no flat surface. The wall surface of a well of a cell-holding part generally has, on the bottom wall side of the culture dish, the above-mentioned concave surface and preferably a conical or circular cone-shaped portion. The wall surface of a well of a cell-holding part may have a wall surface perpendicular to the bottom wall of the culture dish, on the side of the opening from the above-mentioned concave surface.

When the wall surface of a well of a cell-holding part contains a conical or circular cone-shaped portion, an angle formed by the center line and the bus line of the conical or circular-shaped cone generally ranges from 89 to 45°, preferably ranges from 88 to 65°, and more preferably ranges from 85 to 80°. With an angle at a certain level or more, cells can be more easily transferred to a desired place (the deepest part where cells should be located) using gravity force as a driving source. With an angle at a certain level or less, reflection and scattering become difficult to take place on the concave surface upon transmission observation using a microscope, so that clear observation images can be obtained.

When a well of a cell-holding part is composed of a bottom surface parallel to the bottom wall of the culture dish and the side surface perpendicular thereto, cells may move within the well to come into contact with the side surface. If photographic images of cells in such state are taken, a problem arises such that extraction of a cell image by outline extraction from the photographic image is difficult. When the wall surface of a well of a cell-holding part has a concave surface that slopes upward from the lowest position to the outer edge of the well or when preferably such well contains a conical or circular cone-shaped portion, cultured cells automatically remain in the bottom portion of the well. If a well has a side surface perpendicular to the bottom wall of the culture dish on the opening side from the concave surface, cells will never remain in contact therewith and outline extraction of the photographic image of cells can be carried out without problem.

Furthermore, the depth of each well of a cell-holding part is not particularly limited. If the depth is too low, cells move during transport of the culture dish or cell division, for example, so that cells might move outside of the well. Thus, the depth is appropriately determined so as to ensure retention of cells within the well. For example, to retain cells within a well, the depth is preferably ⅓ or more and further preferably ½ or more of the greatest diameter of the cells. On the other hand, if the depth is too high, introduction of a culture solution or cells into a well becomes difficult. Hence, the depth is appropriately determined so that it is not too deep while retaining cells within the well. For example, the upper limit of the depth can be 3 times or less the diameter of the opening of the well. Moreover, to facilitate introduction of a culture solution, the depth is preferably equal to or less than, and particularly preferably ½ or less as great as the diameter of the well. Also, the lower the diameter and the greater the depth of each well, the more difficult it is for the convection flow to take place. Hence, in association with cell respiration and metabolism, composition changes of the surrounding culture solution may take place more easily. Ease of cell growth is altered because of the effects of the composition of the surrounding culture solution. Thus, the diameter and the depth are preferably determined in view of biological influence, so as to accelerate the cell growth. In general, the depth of each well preferably ranges from approximately 50 μm to 200 μm, but may differ depending on cell types. For example, in the case of a bovine embryo, the greatest diameter is approximately 250 μm, so that the depth is preferably 80 μm or more and further preferably 125 μm or more. In addition, the depth refers to a depth that is vertically measured from the opening to the deepest part of a well having a concave surface and if present a side surface perpendicular to the bottom wall of the culture dish.

Four or more wells formed adjacent to each other may be separated by an inner wall surrounding them from the other area within the culture dish. In this embodiment, every group of wells (cell-holding part) adjacent to each other is surrounded by an inner wall. When a plurality of well groups are present on the bottom wall of the culture dish, each group is surrounded by an inner wall. In general, when embryos or the like are cultured, droplets of medium containing embryos are formed in a culture dish and then the droplets are covered with oil, so as to prevent the medium from drying. A group of 4 or more wells formed adjacent to each other is further surrounded by an inner wall, so that the culture dish can hold the medium at the center and can avoid dispersion of the medium. The same also applies when medium is coated with oil such as mineral oil.

Moreover, the culture dish of the present invention may also have a liquid-holding part at a part that is the periphery of the culture dish and has no cell-holding part. That is, an outer moat capable of holding liquid is present on the peripheral side from a portion where the cell-holding part exists, which is formed of another inner wall and a side wall of the culture dish. The interior portion (central space) of the inner wall surrounding wells adjacent to each other can hold medium similarly to the above, the space inside (near the central space) the outer moat can hold oil such as mineral oil, and the outer moat can hold medium, water, or the like. The capacity of the outer moat is generally 1 ml or more. The outer moat holds medium or water, so that it can be used for washing pipettes or glass capillaries when cells are introduced into wells or the humidity within an incubator can be increased. Therefore, the use of mineral oil is not always required for preventing medium from drying.

Also, 4 or more wells formed adjacent to each other may have grooves through which media held in the interior portions are communicable to each other. The presence of such grooves enables circulation of medium throughout wells during cell culture and can enhance the growth of cells each held within a well because of autocrine • paracrine effects.

Material quality of the culture dish of the present invention is not particularly limited. Specific examples of such material include inorganic materials such as metal, glass, and silicon, and organic materials represented by plastic (e.g., a polystyrene resin, a polyester resin, a polyethylene resin, a polypropylene resin, an ABS resin, nylon, an acryl resin, a fluorine resin, a polycarbonate resin, a polyurethane resin, a methyl pentene resin, a phenol resin, a melamine resin, an epoxy resin, and a vinyl chloride resin). The culture dish of the present invention can be produced by methods known by persons skilled in the art. For example, when a culture dish made of a plastic material is produced, the culture dish can be produced by a conventionally used molding method, such as injection molding.

The culture dish of the present invention may be subjected to surface treatment or surface coating so as to accelerate the development of embryos. Particularly when embryos are co-cultured with cells of another organ (e.g., endometrial cells or oviductal epithelial cells) in order to accelerate the development of the embryos, these cells should be caused to adhere to the culture dish in advance. In such case, it is advantageous to coat the surface of the culture dish with a cell adhesive material.

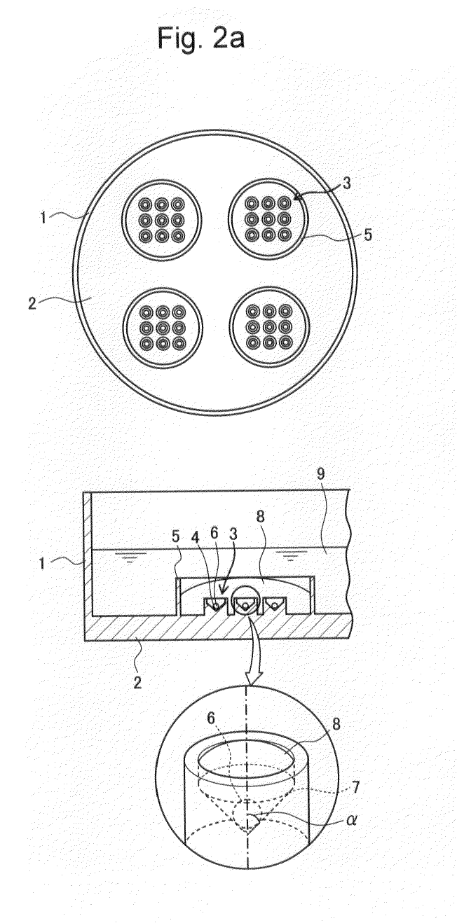

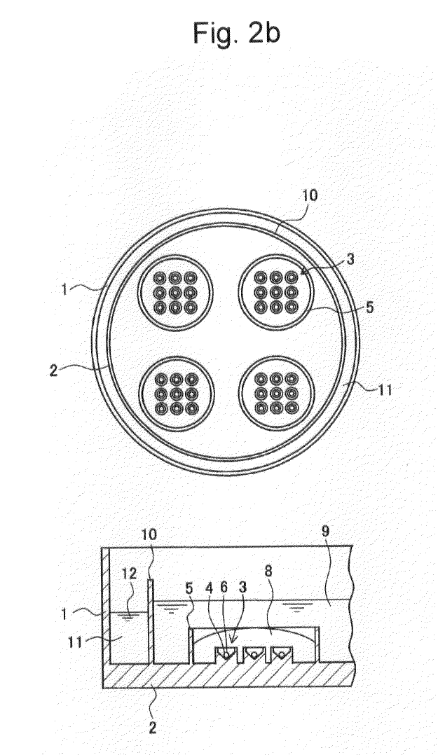

FIG. 2a shows an embodiment of the culture dish of the present invention. FIG. 2a shows an embodiment as follows. The wall surface of each well of the cell-holding parts has a conical (with a rounded top) portion on the bottom wall side of the culture dish. Each well has a wall surface perpendicular to the bottom wall of the culture dish, near the opening thereof. As shown in FIG. 2a, the culture dish has a side wall 1 and a bottom wall 2. On the bottom wall 2, 4 groups of cell-holding parts 3, each consisting of 9 wells 4 (formed adjacent to each other), are formed. Each well 4 holds a cell 6 and has a conical portion, wherein the wall surface (conical surface) 7 slopes upward to the outer edge 13 of the well. An angle formed by the center line and the bus line of the cone is represented by α. In the embodiment of FIG. 2a, groups each consisting of 9 wells adjacent to each other are separated by inner walls 5 surrounding them from the other area within the culture dish. The interior of the inner walls 5 holds medium 8 and the whole culture dish holds mineral oil 9.

FIG. 2b shows another embodiment of the culture dish of the present invention. FIG. 2b shows an embodiment as follows. The wall surface of each well of the cell-holding parts has a conical (with a rounded top) portion on the bottom wall side of the culture dish. Each well has a wall surface perpendicular to the bottom wall of the culture dish, near the opening thereof. As shown in FIG. 2b, the culture dish has a side wall 1 and a bottom wall 2. On the bottom wall 2, 4 groups of cell-holding parts 3, each consisting of 9 wells 4 (formed adjacent to each other), are formed. Each well 4 holds a cell 6 and has a conical portion, wherein the wall surface (conical surface) 7 slopes upward to the outer edge 13 of the well. In the embodiment of FIG. 2b, groups each consisting of 9 adjacent wells are separated by inner walls 5 surrounding them from the other area within the culture dish. Moreover, a liquid-holding part exists on the periphery of the culture dish, where no cell-holding part exists. Specifically, such a form contains an outer moat 11 that is formed of another inner wall 10 and the side wall of the culture dish, on the periphery of the culture dish. The interior (central space) of the inner walls surrounding wells (adjacent to each other) holds medium 8, the interior (central space) surrounded by the outer moat holds mineral oil 9, and the outer moat 11 holds medium or water 12. The use of mineral oil 9 may be omitted. In addition, in FIG. 2a and FIG. 2b, adjacent wells are arranged in the form of a square grid.

The culture dish of the present invention is particularly preferably used when cultured cells are automatically identified. Identification of cultured cells includes identification of the quality of cultured cells. Identification of the qualities of cultured cells involves, when embryos are cultured, for example, identifying whether or not they are high-quality embryos appropriate for implantation into the uterus.

For automated identification of cultured cells, photographic images of cells within the culture dish, which are obtained via a microscope, are taken using a detector such as a CCD camera, the thus obtained images are subjected to outline extraction, portions corresponding to cells in the images are extracted, and then the extracted images of cells are analyzed by an image analyzer, so that the quality can be identified. For outline extraction of images, treatment described in JP Patent Publication (Kokai) No. 2006-337110 A can be used, for example.

Accordingly, in an embodiment, the present invention relates to a method for identifying cultured cells. The identification method of the present invention comprises introducing and culturing cells selected from the group consisting of embryos, ova, ES cells, and iPS cells into or in wells of cell-holding parts of the culture dish of the present invention, taking photographic images of cultured cells obtained via a microscope using a detector such as a CCD camera, and then subjecting the thus obtained images to outline extraction.

In general, cells are cultured by placing a culture dish in an incubator that provides an environmental atmosphere containing gases required for the development and maintenance of cultured cells and certain environmental temperatures. Examples of such required gases include water vapor, free oxygen (O₂), and carbon dioxide (CO₂). Through regulation of environmental temperatures and CO₂ content, the pH of medium can be stabilized within a given length of time. Stable pH can be obtained by stable CO₂ content and stable temperature. Medium for culturing is not particularly limited, as long as it is capable of culturing cells. An example of a culture solution for culturing embryos is M16. An image of a cell during culture is compared with previously stored images with the use of an image comparison program, so that culture conditions of temperature, gas, medium, and the like during culture can also be regulated.

For identification of cultured cells, a method described in JP Patent No. 3693907 can be employed, for example.

The present invention will be described in reference to Examples, but the present invention is not limited within the scope of the Examples.

### EXAMPLES

### Production Example 1

### Production of Culture Dish A

A culture dish shown in FIG. 2a was produced by general injection molding processing. First, a template for molding and producing the culture dish shown in FIG. 2a was processed. A polystyrene material heated and melted was poured onto the template, cooled, and then released from the mold, so that the culture dish A with sloping portions was obtained. The culture dish A with sloping portions was sterilized by placing it under a UV light within a Bio Clean bench for approximately 40 minutes and then used for culturing.

### Production Example 2

### Production of Culture Dish B

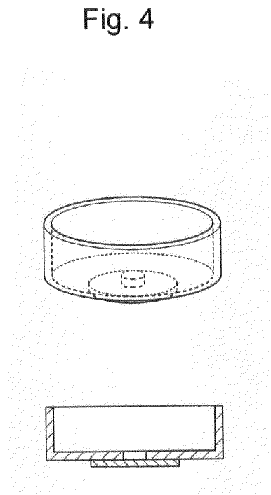

A polystyrene dish with a diameter of approximately 35 mm was prepared. Through-holes with a diameter of 0.4 mm were processed and provided on the bottom surface of the polystyrene dish at intervals of 1 mm, so that a dish with such through-holes was obtained. Next, a cover glass was caused to adhere to the bottom surface of the dish with through-holes from the outside of the dish, so that a culture dish B was obtained (FIG. 4). An adhesive was applied to the lateral side surfaces of the through-holes for adhesion of the cover glass. The culture dish B was sterilized by placing it under a UV light within a Bio Clean bench for approximately 40 minutes, and then used for culture.

### Production Example 3

### Production of Culture Dish C

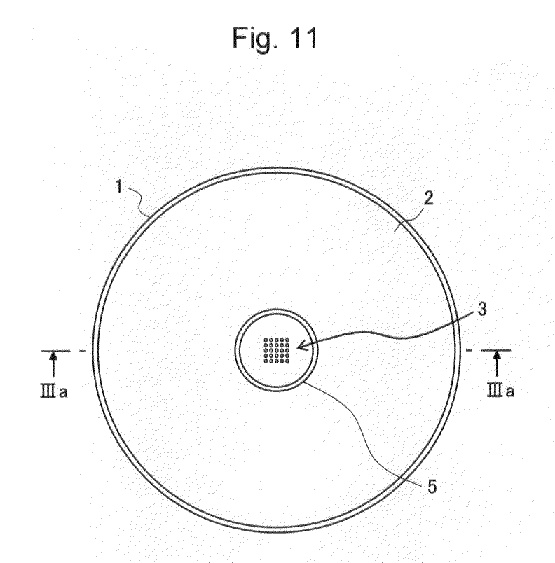

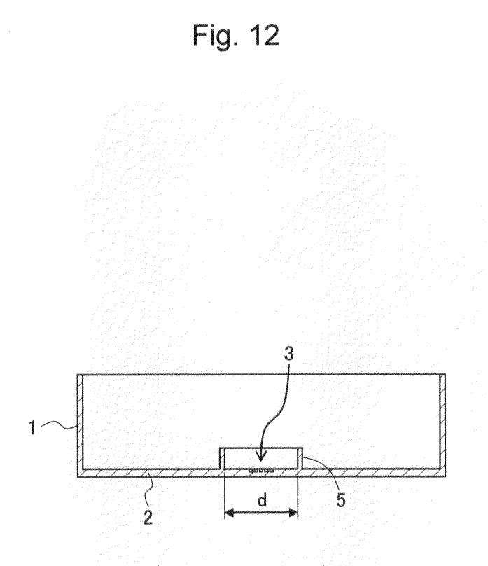

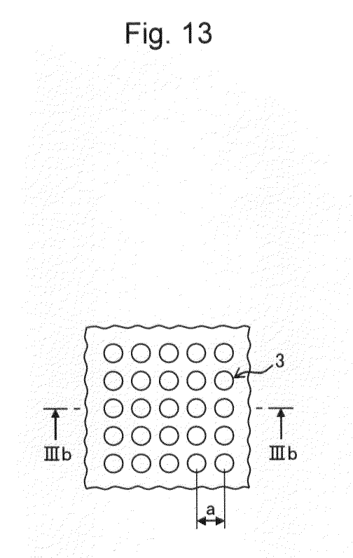

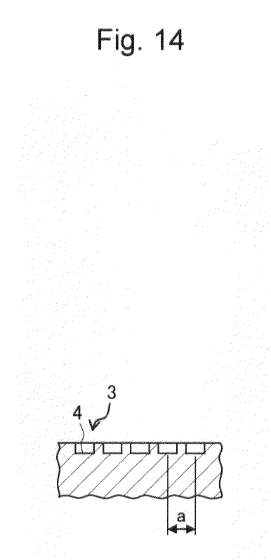

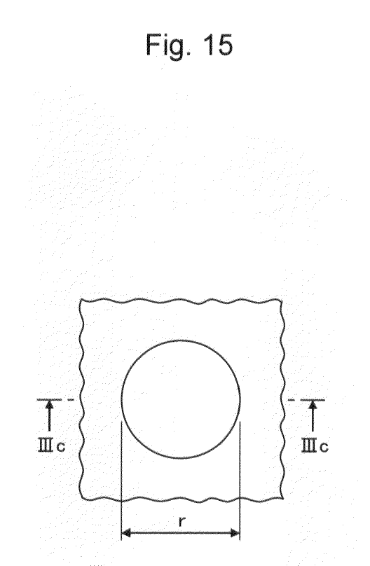

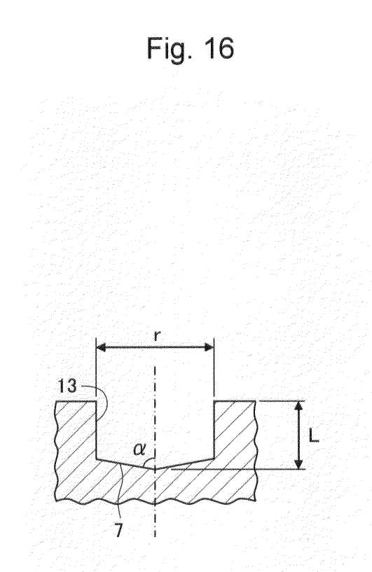

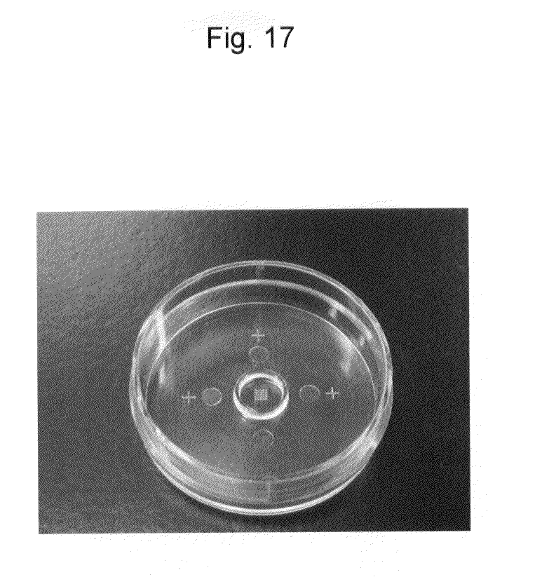

A culture dish C shown in FIGS. 11 to 16 was produced by general injection molding processing. First, a template for molding the culture dish C shown in FIG. 11 was processed and then the smoothness was enhanced by polishing. A polystyrene material heated and melted was poured onto the template, cooled, and then released from the mold, so that the culture dish C with sloping portions was obtained. On the cell-holding part 3 of the culture dish C, 25 wells 4 were formed, which were arranged in the form of a square grid (5×5). A photographic image of the appearance of the thus produced culture dish C is shown in FIG. 17. The culture dish C with sloping portions was immersed in ethanol for 1 hour, washed, and then sterilized by placing it under a UV light within a Bio Clean bench for approximately 40 minutes, and then used for culture. In addition, in FIG. 12, diameter “d” of the region surrounded by the inner wall 5 is 7 mm and the pitch “a” of wells 4 shown in FIGS. 13 and 14 is 420 μm. Also in FIGS. 15 and 16, the diameter “r” of each well was determined at 270 μm and the depth “L” was determined at 150 μm. Furthermore, regarding conically-formed wall surface 7, the angle α formed by the center line and the bus line of the cone was determined at 83°.

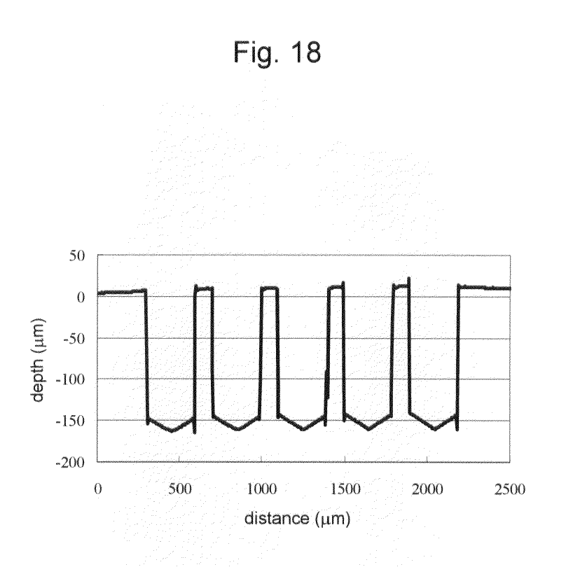

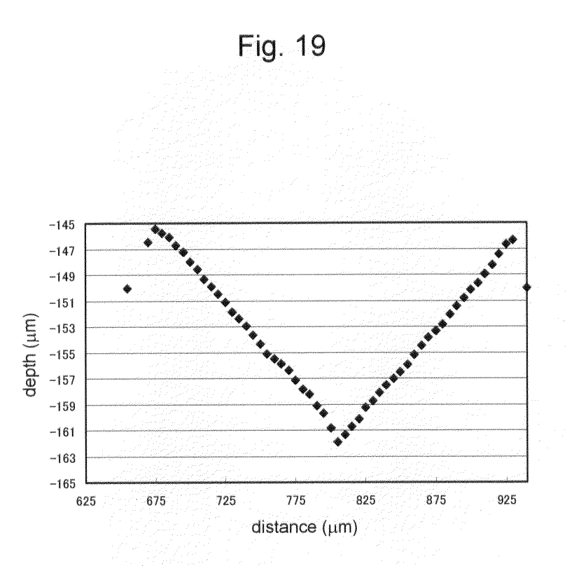

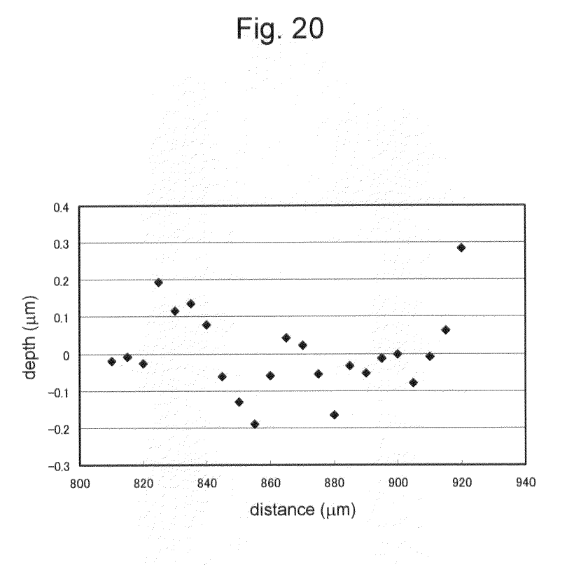

FIG. 18 shows the results of measuring the cross section of the cell-holding part 3 of the thus produced culture dish C (measurement instruments: KEYENCE CORPORATION, double scan high-precision laser measuring apparatus LT-9010M and high-precision shape measuring system KS-1100, at intervals of 5 μm). FIG. 19 shows the results of measuring the cross section of the wells 4 (measurement instruments and measurement conditions are similar to those described above). Also, FIG. 20 shows a graph showing the results of correcting the slope by carrying out linear fitting for the concave surface on the right in FIG. 19. Based on FIG. 20, the maximum height “Ry” was found to be 0.47 μm.

### Production Example 4

### Production of Culture Dish D

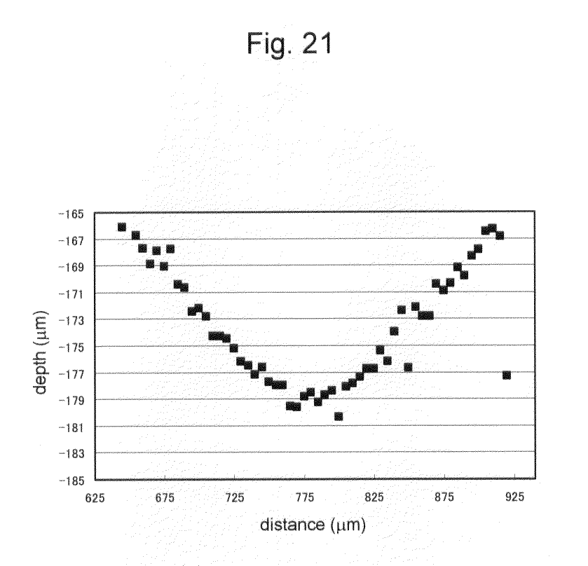

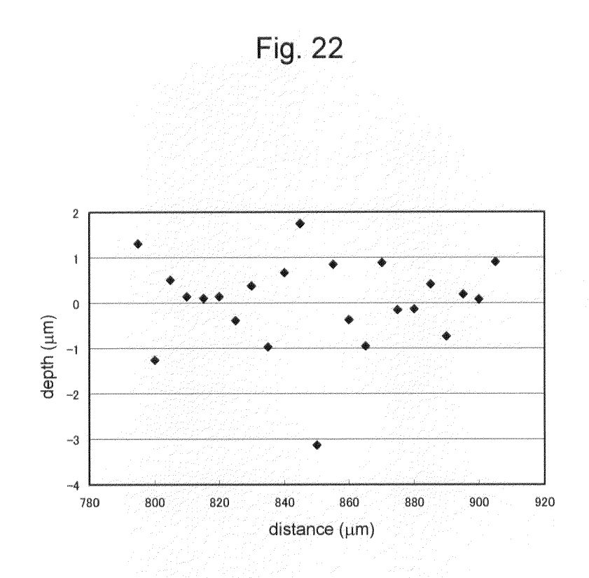

At the time of template production, a culture dish D was produced with procedures similar to those in the production example 3 above except that no polishing was carried out. FIG. 21 shows the results of measuring the cross sections of wells 4. Also, FIG. 22 shows the results of further correcting the slope by carrying out linear fitting for the concave surface on the right in FIG. 21. Based on FIG. 22, the maximum height “Ry” was found to be 4.9 μm.

### Production Example 5

### Production of Culture Dish E

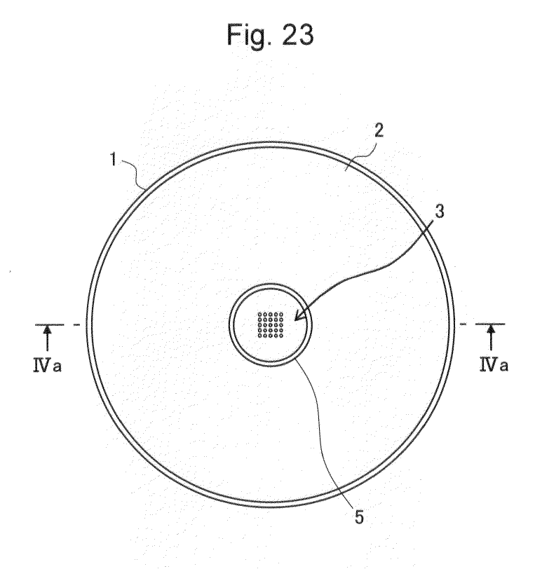

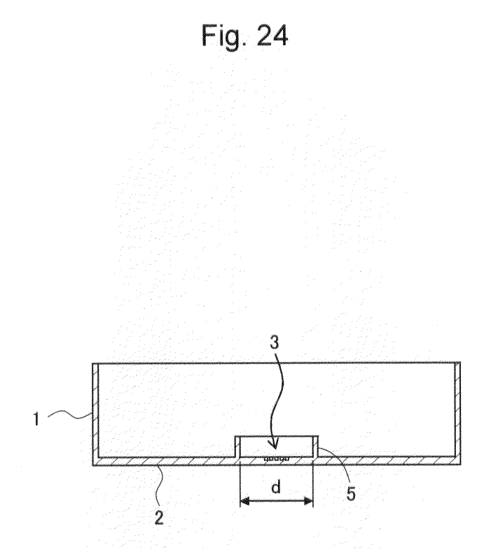

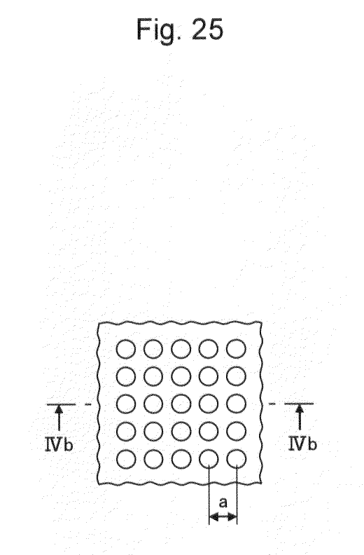

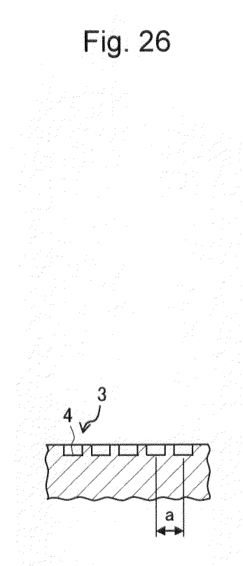

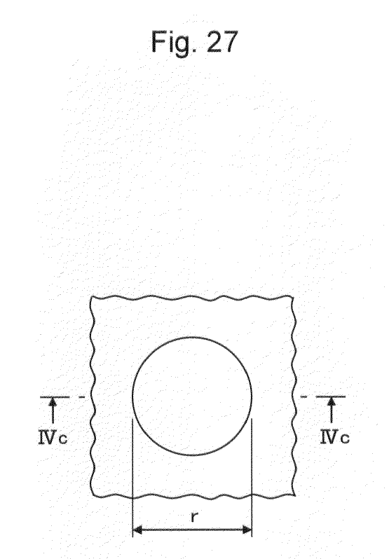

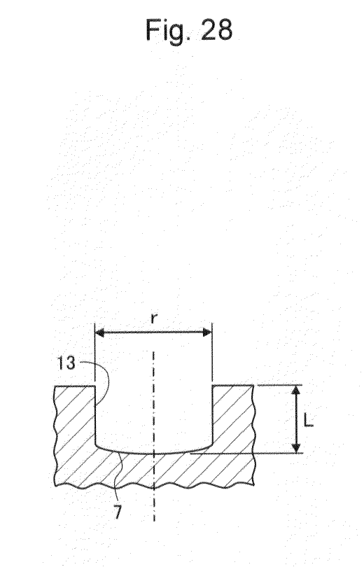

A culture dish E shown in FIG. 23 to FIG. 28 was produced by procedures similar to those in Production example 3 above. In addition, in the culture dish E, the wall surface 7 of each well 4 is formed to be a concave surface that is curved upward from the deepest part to the outer edge 13 of the well. The other dimensions of the culture dish E are the same as those of the culture dish C shown in FIGS. 11 to 16.

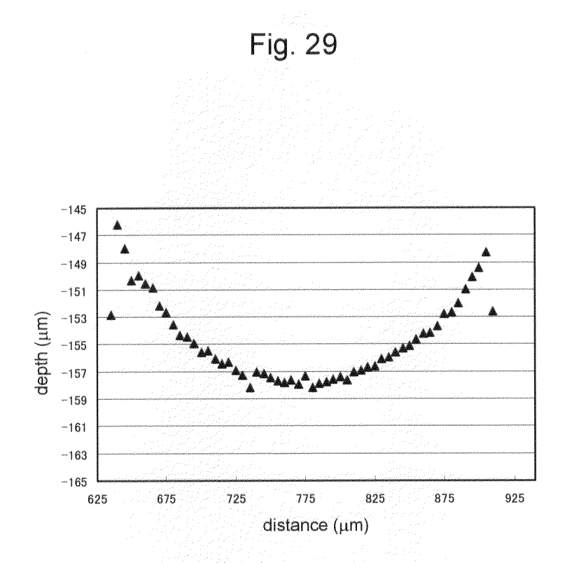

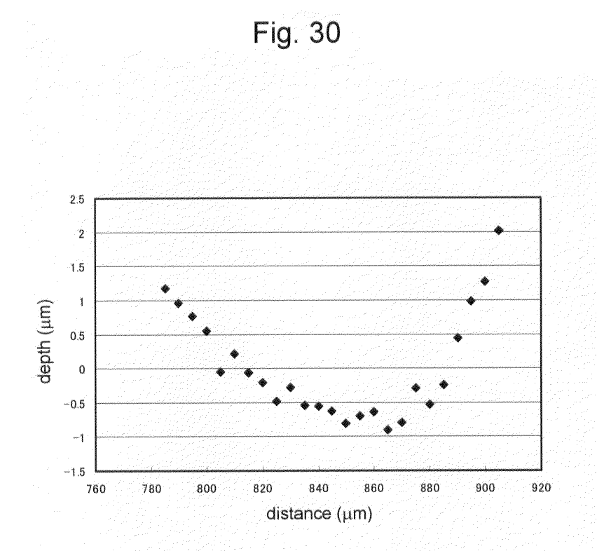

FIG. 29 shows the results of measuring the cross section of each well 4 of the culture dish E. Also, FIG. 30 shows the results of correcting the slope by carrying out linear fitting for the concave surface on the right in FIG. 29. Based on FIG. 30, the maximum height “Ry” was found to be 2.9 μm.

### Example 1

Rat embryos were cultured using the culture dish A produced in Production example 1 and then observed. The images of embryos were subjected to outline extraction.

Rat embryos were introduced using a glass capillary into wells of cell-holding parts of the culture dish A in Production example 1. Furthermore, mineral oil was poured so as to cover the medium to prevent evaporation thereof. Rat embryos were cultured using a CO₂ incubator (5% CO₂, 5% O₂, and 90% air, 37° C., saturated humidity).

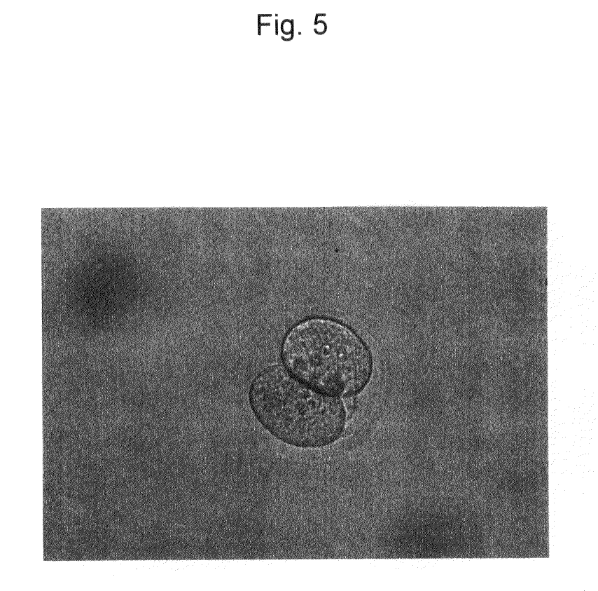

After initial cleavage of an embryo, a photographic image thereof was taken using a 20-power observation apparatus (provided with lens of high power and an imaging camera). The photographic image is shown in FIG. 5.

For extraction of the outline of the embryo, threshold processing was performed as follows.

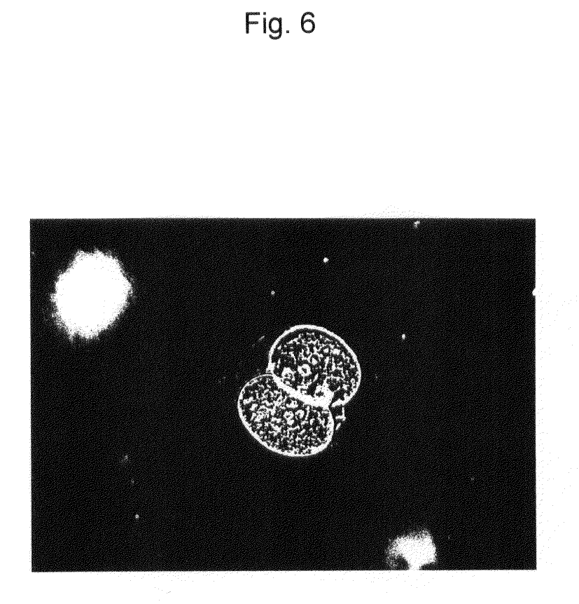

FIG. 6 shows an image obtained by performing threshold processing for the image of FIG. 5. When the culture dish A of Production example 1 was used, as a result of threshold processing, the outline of the embryo could be recognized without being affected by the wall surface of the well, as shown in FIG. 6.

Threshold processing (=binarization) involves defining 1 (white) and 0 (black) when the value of a picture element (=brightness level) of each photographic image of an embryo taken by a camera is a value between a previously determined first threshold (or greater) and a previously determined second threshold (or less) (first threshold<second threshold), for example. Threshold processing also involves defining 0 (black) and 1 (white) when the value of a picture element of the same is a value that is less than the 1^{st} threshold or greater than the 2^{nd} threshold.

### Example 2

Mouse embryos were cultured using the culture dish C produced in Production example 3 and then observed. The images of embryos were subjected to outline extraction.

The mouse embryos were introduced using a glass capillary into wells of the cell-holding part of the culture dish C in Production example 3. Furthermore, mineral oil was poured so as to cover the medium to prevent evaporation thereof. Mouse embryos were cultured using a CO₂ incubator (5% CO₂, 5% O₂, and 90% air; 37° C., saturated humidity).

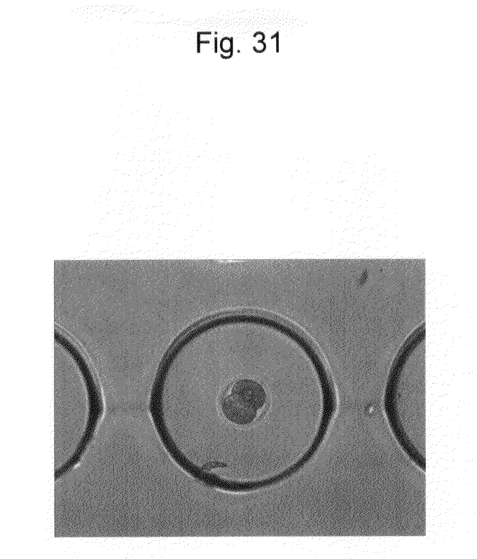

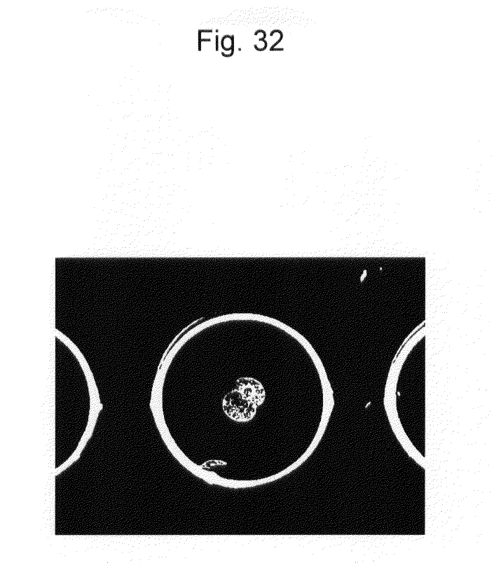

After initial cleavage of an embryo, a photographic image thereof was taken using a 10-power observation apparatus (provided with lens of high power and an imaging camera). The photographic image is shown in FIG. 31. Threshold processing was performed similarly to Example 1, so that the outline of the embryo was extracted. FIG. 32 shows an image obtained by performing threshold processing for the image of FIG. 31. When the culture dish C in Production example 3 was used, as a result of threshold processing, the outline of the mouse embryo could be recognized without being affected by the wall surface of the well, as shown in FIG. 32.

### Example 3

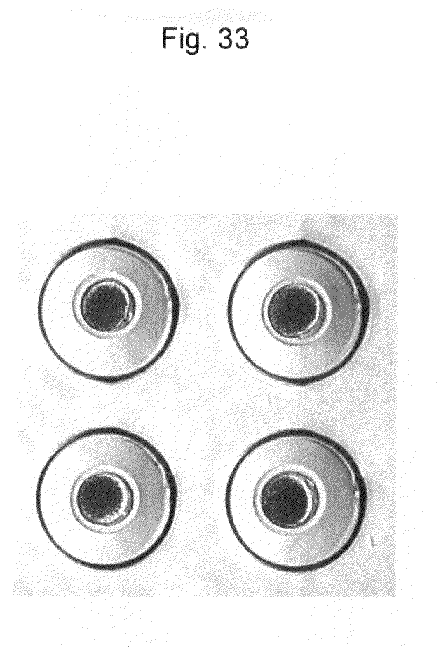

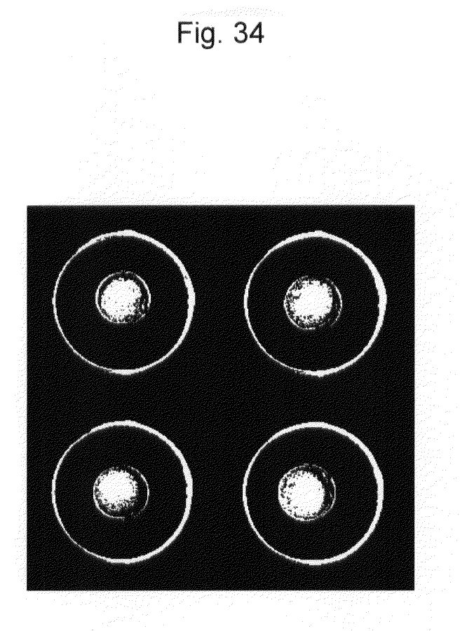

Immediately after introduction of bovine embryos into the culture dish C similarly to Example 2, a photographic image of a bovine embryo was taken using a 4-power observation apparatus (provided with lens of high power and an imaging camera). A portion extracted from the photographic image is shown in FIG. 33. The outline of the embryo was extracted by threshold processing similar to Example 1. FIG. 34 shows an image obtained by performing threshold processing for the image of FIG. 33. When the culture dish C in Production example 3 was used, as a result of threshold processing, the outline of the bovine embryo could be recognized without being affected by the wall surface of the well, as shown in FIG. 34.

### Example 4

Mouse embryos were introduced into the culture dish D similarly to Example 2. After initial cleavage, photographic images thereof were taken using a 10-power observation apparatus (provided with lens of high power and an imaging camera). Threshold processing was performed similarly to Example 1, so that the outline of each embryo was extracted. The outline of the mouse embryo could be recognized without being affected by the wall surface of the well. However, the concave surface of each well of the cell-holding part was not smoothed, resulting in low transmittance, affection by noise, and recognition more difficult than that in Example 2.

### Example 5

Mouse embryos were introduced into the culture dish E similarly to Example 2. After initial cleavage, photographic images thereof were taken using a 10-power observation apparatus (provided with lens of high power and an imaging camera). When the outline of an embryo was extracted by threshold processing similar to that in Example 1, the outline of the mouse embryo could be recognized without being affected by the wall surface of the well. However, the concave surface of each well of the cell-holding part was not linear, unlike the culture dish C, but had curvature. Hence, the probability of embryos being in contact with the wall surface of wells was higher than that in Example 2. Based on the results of Examples 2, 4, and 5 above, it was demonstrated that the concave surface of each well containing a straight section and having as low a value of “Ry” as possible is capable of keeping cells at the center of each well, so that it is advantageous in identification of cells.

### Comparative Example 1

In Comparative example 1, mouse embryos were cultured using the culture dish B produced in Production example 2 and then observed. The images of the embryos were subjected to outline extraction.

Mouse embryos were introduced using a glass capillary into wells of a cell-holding part of the culture dish B in Production example 2. Furthermore, mineral oil was poured so as to cover the medium to prevent evaporation thereof. Mouse embryos were cultured using a CO₂ incubator (5% CO₂, 5% O₂, and 90% air; 37° C., saturated humidity).

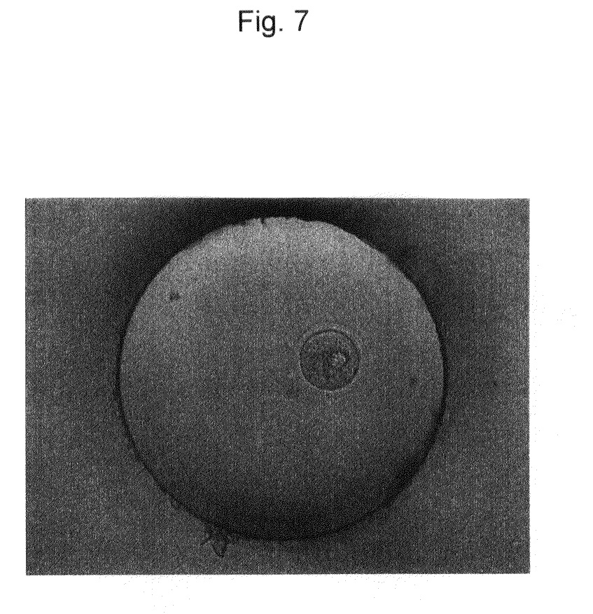

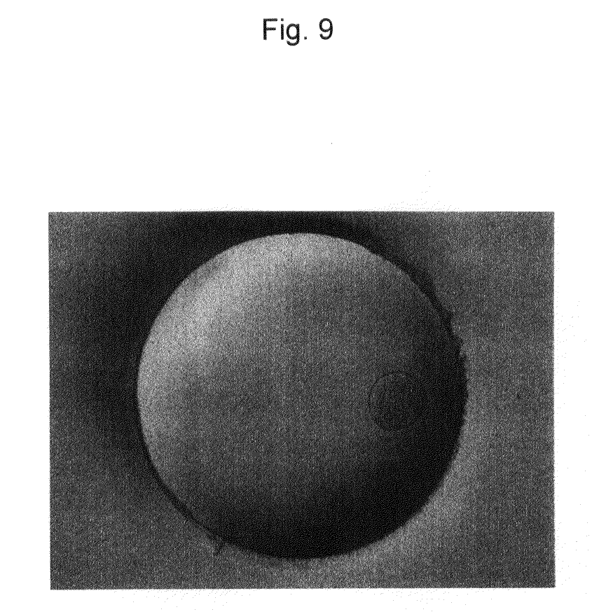

Immediately after introduction of embryos into the culture dish B produced in Production example 2, photographic images thereof were taken using a 10-power observation apparatus (provided with lens of high power and an imaging camera). The photographic images are shown in FIGS. 7 and 9.

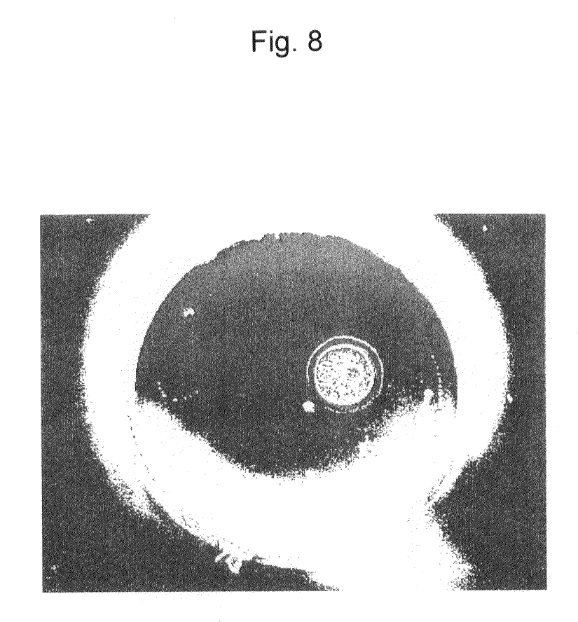

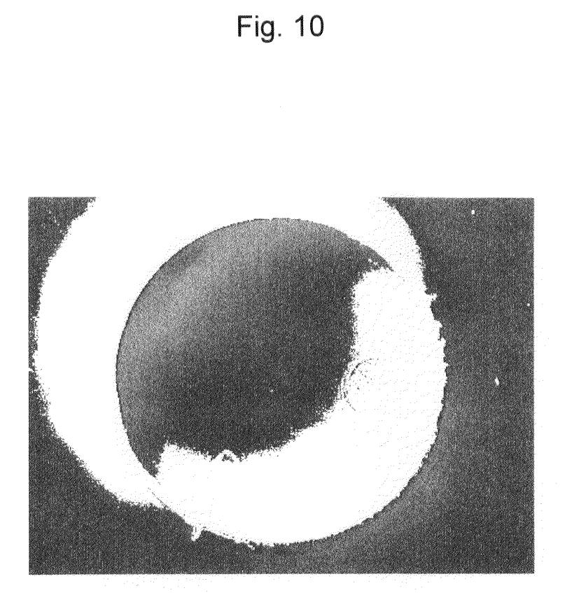

The outline of each embryo was extracted by threshold processing similar to that in Example 1. FIG. 8 shows an image obtained by performing threshold processing for the image of FIG. 7 and FIG. 10 shows an image obtained by performing threshold processing for the image of FIG. 9.

As in FIG. 8, the outline of an embryo that was accidentally placed away from the wall surface of a well of the cell-holding part could be extracted. However, as in FIG. 10, the outline of an embryo that was in contact with the wall surface of a well of the cell-holding part could not be extracted.

### EXPLANATION OF LETTERS OR NUMERALS

1: side wall, 2: bottom wall, 3: cell-holding part, 4: wells of a cell-holding part(s), 5: inner wall, 6: cell, 7: wall surface (conical surface) of a well, 8: medium, 9: mineral oil, 10: inner wall, 11: outer moat, 12: medium or water, 13: outer edge

1. A culture dish for culturing cells that require separate control, having a bottom wall and a side wall, wherein:
a cell-holding part having wells is arranged on the bottom wall;
4 or more wells are adjacent to each other;
the wall surface of each well has a concave surface that slopes upward from the lowest position to the outer edge of the well; and
the pitch between wells adjacent to each other is 1 mm or less.

2. The culture dish according to claim 1, wherein the wall surface of each well has a concave surface having a straight section.

3. The culture dish according to claim 2, wherein the surface roughness of the concave surface is characterized in that the maximum height Ry is less than 1.0 μm.

4. The culture dish according to claim 1, wherein the width of the opening of each well ranges from 100 μm to 300 μm.

5. The culture dish according to claim 1, wherein the depth of each well ranges from 50 μm to 200 μm.

6. The culture dish according to claim 1, wherein the opening of each well is circular.

7. The culture dish according to claim 6, wherein the wall surface of each well has a conical or circular cone-shaped part.

8. The culture dish according to claim 7, wherein an angle formed by the center line and the bus line of the conical or circular cone-shaped part ranges from 89° to 45°.

9. The culture dish according to claim 1, wherein the wells adjacent to each other are arranged at a density of at least one well per 1 mm².

10. The culture dish according to claim 1, wherein 4 or more wells adjacent to each other are arranged in the form of a square grid or close packing.

11. The culture dish according to claim 1, wherein 24 or more wells are arranged.

12. The culture dish according to claim 1, wherein the cell is selected from the group consisting of an embryo, an ovum, an ES cell, and an iPS cell.

13. The culture dish according to claim 12, wherein the cell is a bovine embryo.

14. The culture dish according to claim 1, wherein 4 or more wells adjacent to each other are separated by an inner wall surrounding the wells from the other part within the culture dish.

15. The culture dish according to claim 14, having a liquid-holding part that is a peripheral part of the culture dish having no cell-holding part.

16. The culture dish according to claim 1, which is used for automatically identifying cultured cells.

17. The culture dish according to claim 16, regarding which a visual field for observation contains 4 or more wells when a cell-holding part is observed via a microscope using a 4× objective lens.

18. A method for identifying cultured cells, comprising
introducing and culturing cells selected from the group consisting of embryos, ova, ES cells, and iPS cells in wells of a cell-holding part of the culture dish according to claim 1,
taking photographic images of cultured cells obtained via a microscope using a detector, and subjecting the thus obtained images to outline extraction.
